(19) United States
(12) Patent Application Publication
(10) Pub. No.: US 2025/0259703 A1
(43) Pub. Date: Aug. 14, 2025

(54) INFERRING CLONAL POPULATION STRUCTURE USING MULTILEVEL GENETIC ALGORITHMS FOR MEDICAL DECISION MAKING

(71) Applicant: NEC Laboratories America, Inc., Princeton, NJ (US)

(72) Inventors: Jonathan Warrell, Princeton, NJ (US); Francesco Alesiani, Heidelberg (DE); Anja Moesch, Munich (DE); Renqiang Min, Princeton, NJ (US)

(21) Appl. No.: 19/050,288

(22) Filed: Feb. 11, 2025

Related U.S. Application Data
(60) Provisional application No. 63/552,740, filed on Feb. 13, 2024.
(60) Provisional application No. 63/651,418, filed on May 24, 2024.

(51) Int. Cl. G16B 20/20; G06N 20/00; G16B 20/40; G16H 20/17

(52) U.S. Cl. CPC G16B 20/20; G06N 20/00; G16B 20/40; G16H 20/17

(57) ABSTRACT

The present disclosure relates to medical and health decision making and, more particularly, to treatment based on tumor clonality estimates. Methods and systems include analyzing genotypes of a tumor to identify clonality sub-types present in the tumor, using a machine learning model that is trained to learn a multilevel evolutionary process or genetic algorithm, by using a recursive Wasserstein objective to output the clonal sub-types, an ancestral structure, and a fitness model. A treatment is generated, tailored to the tumor using the clonal sub-types, and subclonal properties predicted by the model, such as subclone fitness.

### RELATED APPLICATION INFORMATION

This application claims priority to U.S. Patent Application No. 63/552,740, filed on Feb. 13, 2024, and to U.S. Patent Application No. 63/651,418, filed on May 24, 2024, each incorporated herein by reference in its entirety.

### BACKGROUND

### Technical Field

The present invention relates to medical decision making and, more particularly, to treatment based on tumor clonality estimates.

### Description of the Related Art

Ancestry trees grow super-exponentially with the size of a population. As a result, inference of ancestry structure in population genetics can be challenging. Attempting to infer individual population trees from genetics data, given a fixed mutation model and the assumption of neutrality (no fitness effects) for most variants, as is typically assumed in models of cancer, neglects the interaction of aggregated fitness effects of large numbers of variants with the individual tree structures.

### SUMMARY

A method includes analyzing genotypes of a tumor to identify clonality sub-types present in the tumor, using a machine learning model that is trained to learn a multilevel evolutionary process or genetic algorithm, by using a recursive Wasserstein objective to output the clonal sub-types, an ancestral structure, and a fitness model. A treatment is generated, tailored to the tumor using the clonal sub-types, and subclonal properties predicted by the model, such as subclone fitness.

A system includes a hardware processor and a memory that stores a computer program. When executed by the hardware processor, the computer program causes the hardware processor to analyze genotypes of a tumor to identify clonality sub-types present in the tumor, using a machine learning model that is trained to learn a multilevel evolutionary process or genetic algorithm, by using a recursive Wasserstein objective to output the clonal sub-types, an ancestral structure, and a fitness model, and to generate a treatment tailored to the tumor using the clonal sub-types.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram of a multi-level evolutionary process, in accordance with an embodiment of the present invention;

FIG. 2 is a block/flow diagram of a method for tailoring a treatment to a patient based on a multi-level evolutionary analysis, in accordance with an embodiment of the present invention;

FIG. 3 is a block diagram of a healthcare facility that tailors treatments based on evolutionary analysis, in accordance with an embodiment of the present invention;

FIG. 4 is a block diagram of a computing device that has a model which is trained to tailor treatments based on an evolutionary analysis, in accordance with an embodiment of the present invention;

FIG. 5 is a diagram of an exemplary neural network architecture that can be used to implement a model for evolutionary analysis, in accordance with an embodiment of the present invention; and

FIG. 6 is a diagram of an exemplary deep neural network architecture that can be used to implement a model for evolutionary analysis, in accordance with an embodiment of the present invention.

### DETAILED DESCRIPTION OF PREFERRED EMBODIMENTS

Ancestry structure may be jointly inferred using tree structure and a fitness model over multiple populations (a meta-population), allowing the fitness model to inform the inference of individual trees for individual populations. Information may be drawn at the genomic and epigenomic levels in addition to the genetics level to help infer fitness functions in individual applications.

In particular tumor cell clonality may be inferred for personalized cancer treatment. The number and composition of subclones in a tumor can be used to select a treatment and/or vaccine development. For example, the presence of particular subclones may predict whether a patient will respond to a particular treatment. Further, an effective cancer vaccine would target peptides from all high-risk clones in a cancer. In vitro experimental data can further be used to inform the development of informative clonal fitness models.

To that end, a machine learning model is trained using an objective function that is based on a recursive application of the Wasserstein distance, to jointly infer tree structures and a fitness model over multiple populations. The recursive objective function may be extended to an indefinite number of levels and may model multi-level processes in biology. For the particular application of tumor clonality inference, two or three levels may be used, corresponding to the cellular and tumor levels, or the cellular, tumor region (primary, secondary etc.) and tumor levels respectively. The machine learning model may be a neural network or generalized linear model that predicts the fitness of a tumor cell from genetic and/or epigenetic data.

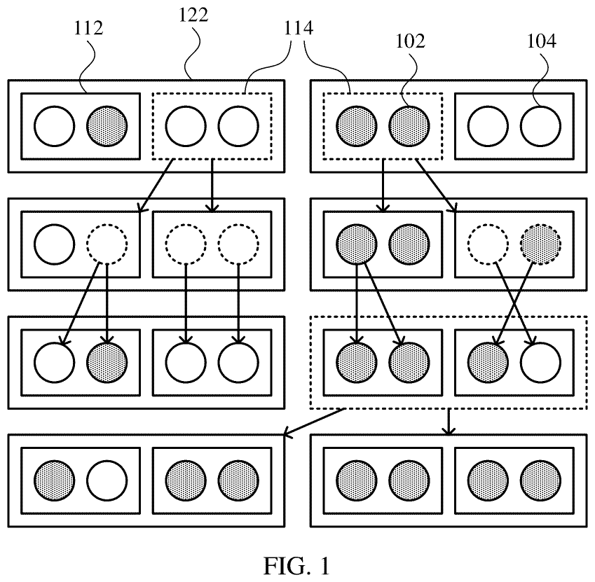

Referring now to FIG. 1, a diagram of a multi-level evolutionary process is shown. A population includes L=3 levels. The first level l=0 is the level of individual members, which are shown as exhibiting two genotypes, indicated by shaded members 102 and unshaded members 104. The second level l=1 is made up of pairs of members, indicated by boxes 112. Four such pairs 112 are shown. The third level l=2 is made up of meta-groups that each include two pairs 112, indicated by boxes 122. Thus the meta-groups include four members each.

Time t flows downward. At each time step, a level is selected to reproduce and the individuals (or groups) at that level which reproduce are shown in dotted lines. Thus, in the first time step, two pairs 114 are selected for reproduction to generate the population of the next level. Mutations may occur during reproduction, for example changing a particular member from shaded to unshaded or from unshaded to shaded. When a group at level I reproduces, the Wasserstein distance at level I determines the probability for the state of the offspring group. The fitness of a group determines its probability of reproducing and is determined by the fitness of the group members and the group's cohesion. For example, the shaded members may have a higher level-0 fitness than the unshaded members.

An evolutionary process exhibits multilevel selection when it operates on units at multiple levels, each with its own definition of fitness. For example, an evolutionary process involving ant colonies will exhibit three levels, including individual cells within an ant, individual ants, and whole colonies. Major transitions in evolution, such as the emergence of multicellularity, can be modeled in terms of multilevel selection. At a different scale, cancer can be viewed as a multilevel evolutionary process, involving cells, tumor regions, and the tumor itself.

Existing models for multilevel evolutionary processes fail to consider how parameters may be learned from real or synthetic data that is generated by the process. As described below, a model may be implemented for multilevel selection that is minimal, in the sense of including only those components needed to embed a basic level of biological realism. Arbitrarily large genotypes and phenotypes may be embedded, while providing a consistent mechanism for extending the model to an arbitrary number of levels.

The model may be based on the Kantorivich Monad on the category of 1-bounded compact metric spaces and non-expansive maps. Hence the functor [symbol] : [symbol] [symbol] et₁→ [symbol] [symbol] et₁ may be used, taking a metric space (X, d₁) to the metric space ( [symbol] X, d₂), where [symbol] [symbol] is the set of Borel probability measures over X, and d₂ is the Wasserstein distance, defined as:

$${d}_{2} ( μ , v ) = {min}_{τ ∈ {T}_{μ , v}} {\int }_{X \times X} {d}_{1} ( {x}_{1} , {x}_{2} ) d τ ( {x}_{1} , {x}_{2} )$$

is T_{μ,ν} the set of Borel probability measures τ on X×X, such that ∫_{X}τ(x, .)dμ(x)=ν and ∫_{X}τ(., x)dv(x)=μ, and μ, ν ∈ [symbol] [symbol] are two given Borel probability measures. ( [symbol] f)(μ) is defined as the push-forward measure. Additionally, monad unit and multiplication functions are needed. The unit, ηₓ: X→ [symbol] X is defined as ηₓ(x)=δ(,; x), where δ(,; x) is the delta distribution at x and the multiplication μₓ: [symbol] [symbol] X→ [symbol] [symbol] is defined as (μ_{X}ζ)(B)=∫_{X}ν(B)dζ(ν), where B⊂X is a Borel set. With these definitions, ( [symbol] , η, μ) can be shown to satisfy the monad properties.

A meta-population is defined as a higher-order finite discrete distribution over a base space of genotypes (which themselves are not necessarily discrete). Hence, (X, d) denotes a space of genotypes, where d is an arbitrary distance function. Then, a meta-population x is parameterized by setting a level l, and a population size n. A meta-population xₗₙ is thus a member of [symbol] ^{L}X, having the form:

$${x}_{l n} = ( 1 / n ) {\sum }_{1 - 1 … n} δ ( . ; {x}_{l - 1 , n}^{( 1 )} )$$

where x_{i-1,n}⁽ⁱ⁾, i=1. . . n, are n meta-populations at level l−1, and we assume l>0. Meta-populations at level l=0 are simply elements of X (genotypes).

A genetic algorithm may be used. An example of such may be a multilevel Wright-Fisher process, which is defined by fixing a maximum level L and a chosen population size N. Additionally, a fitness function is needed over the genotypes (at level-0), f₀: X→[01], as well as a cooperativity strength, κ, a mutation penalty λ, and

potentially a base conditional distribution Δ₀(y|x) over the genotypes, representing the probability that x mutates to y. The process then defines a Markov chain over meta-populations X_{LN}. Hence, a sample from the process consists of a sequence x_{LN}^{(t-0)}, x_{LN}^{(t-1)}, . . . , x_{LN}^{(t-T)}.To define the dynamics of the process, the fitness function is extended across levels 1 . . . L. For level l, f^{l} defines the fitness across levels 1 . . . L. For level l, f^{l} defines the fitness as:

$${f}_{l} ( {x}_{l} ) = κ \cdot ψ ( {x}_{l} ) + {\sum }_{i = 1 … N} {f}_{l - 1} ( {x}_{l - 1}^{( 1 )} )$$

where the subscripts n are dropped on the meta-populations for convenience (since these are fixed to N at all levels, by definition), and the consistency function, Ψ, is defined as:

$$ψ ( {x}_{l} ) = \frac{{\sum }_{l , j \leq N} ( 1 - {d}_{l} ( {x}_{l - 1}^{( i )} , {x}_{l - 1}^{( j )} ) )}{{N}^{2}} .$$

The dynamics for the meta-population evolution then includes, at each time step, sampling a level l∈0 . . . L−1 to update (either uniformly, or according to a parameterized distribution { [symbol] l=0 . . . L−1}, and then updating the meta-population at level l according to the following transition kernel:

$${P}_{l} ( {x}_{l}^{t} \vert {x}_{l}^{t - 1} ) ∝ {\sum }_{{x}_{l}} {\prod }_{l = 1 … {N}^{L - 1}} \tilde{f} ( {x}_{l}^{t - 1 , π ( t )} ) {Δ}_{l} ( {x}^{t , i} \vert {x}^{t - 1 , π ( t )} )$$

$$where :$$

$$\tilde{f} ( {x}_{l}^{t , i} ) = \frac{f ( {x}_{l}^{t , i} )}{{\sum }_{j ∈ {S}_{i , l}} f ( {x}_{l}^{t - 1 , j} )}$$

and πₗ denotes a mapping of the meta-populations at level l and time t to those at level l and time t−1 (e.g., mapping a child meta-population or genotype to its parent at the previous time-step), part of the same meta-population at level l+1 as i. It remains to define Δₗ(⋅|⋅), the conditional mutation probability function. One possibility is to directly use the Wasserstein distance between meta-populations at level l to define a mutation probability at level l; hence:

$${Δ}_{l} ( {y}_{l} \vert {x}_{l} ) ∝ {e}^{\{ - λ {d}_{l} ( {x}_{i} , {y}_{l} ) \}}$$

While this is the simplest possibility, the constraint that Δₗ(⋅|⋅) be derived from the symmetric distance function dₗ at each level may not always be appropriate. For this reason, a second possibility is that a base distribution Δ₀(⋅|⋅) is provided independently, and for levels l≥1 the number of ‘swaps’ that have occurred between sub-populations at each level is used to generate a chosen meta-population from a given one. For this case, a possible form for Δₗ(⋅|⋅) is:

$${Δ}_{l} ( {y}_{l} \vert {x}_{l} ) ∝ {\sum }_{{g}_{0} , … , {g}_{l - 1}} {\prod }_{{l}^{′} - 1 … l - 2} exp \{ - {\sum }_{i ∈ 1 … {N}^{l - {l}^{′} + 1}} λ [ {g}_{1} ( ⌊ \frac{i}{{N}^{l - {l}^{l}}} \vert ) = \vert \frac{{g}_{{l}^{′} - 1} ( i )}{{N}^{l - {l}^{′}}} ] ] \} \cdot {\prod }_{i = 1 … {N}^{l}} {Δ}_{0} ( {y}_{0}^{( i )} \vert {x}_{0}^{( {g}_{0} ( i ) )} )$$

where [.] is the Iverson bracket, and gy is a permutation over the set {0 . . . N^{l-l′}}. To complete the model specification, an initial distribution over meta-populations may be assumed at t=0 to be P₀(x_{LN}^{t=0}). FIG. 1 illustrates a multi-level evolutionary process.

Various approaches may be used to fit parameters of a multilevel evolutional process, such as variational optimization, simultaneous perturbation stochastic perturbation, and Monte-Carlo expectation maximization. Each method may be used by combining both forward simulations of the model and coalescent (backwards) simulations.

A variational distribution over the parameters θ may be set as a Gaussian with a symmetric covariance matrix. AT a given meta-epoch τ, this variational distribution has the form:

$${θ}_{τ} ∼ ( . \vert {μ}_{τ} , {σ}_{τ} I )$$

where μ_{τ} is a vector of mean values, σ_{τ} is a scalar, and l is the identity matrix. At meta-epoch τ, S samples may be drawn from θ_{τ}, θ_{τ}¹ . . . θ_{τ}^{S}, and for each, R forward multilevel Wright-Fisher processes are run to generate meta-population samples x_{L}^{T,s,r}, for r=1 . . . R. These may be used to calculate the following score:

$${F}_{s}^{τ} = 1 - {𝔼}_{{x}_{L}^{T} ∼ {P}_{θ .}^{L T}} [ {d}_{L} ( {x}_{L}^{T} , {x}_{L}^{T †} ) ] \approx 1 - \frac{1}{R} {\sum }_{r} {d}_{L} ( {x}_{L}^{T , s , r} , {x}_{L}^{T †} )$$

where d_{L}(.,.) is the Wasserstein distance at level L, and P_{θ}^{L,T} is the distribution over [symbol] ^{L}X at time-step T induced by the multilevel Wright-Fisher process with parameters θ.

Smoothing-based optimization (SMO) updates may be applied to update μ and σ:

$${μ}_{τ + 1} = \frac{{\sum }_{s} {F}_{s} {θ}_{τ}^{s}}{{\sum }_{s} {F}_{s}}$$

$${σ}_{τ + 1} = \sqrt{\frac{{\sum }_{s} {F}_{s} {\vert {θ}_{τ}^{s} - {μ}_{τ} \vert}_{2}^{2}}{{D}_{θ} {\sum }_{s} {F}_{s}}}$$

These updates improve the value of F in expectation as S→∞ and R→∞, and hence reduce the expected Wasserstein distance between the simulated and ground truth processes:

$${𝔼}_{θ ∼ {Q}_{τ + 1}} {𝔼}_{{x}_{L}^{T} ∼ {P}_{θ}^{L , T}} [ {d}_{L} ( {x}_{L}^{T} , {x}_{L}^{T †} ) ] \leq {𝔼}_{θ ∼ {Q}_{τ}} {𝔼}_{{x}_{L}^{T} ∼ {P}_{θ}^{L , T}} [ {d}_{L} ( {x}_{L}^{T} , {x}_{L}^{T †} ) ]$$

where Q_{τ}= [symbol] (.|μ_{τ}, σ_{τ}I).

Exclusively using forward simulations may lead to noisy estimates of the expected Wasserstein distance for a given sample θₛ, due to the small likelihood of generating samples close to the ground-truth data. To stabilize the expected Wasserstein distance estimates, it can be assumed that a coalescent sampler [symbol] (.|x_{L}^{T†}, θ) is available, which generates possible meta-population trajectories given a final ground-truth state. Then:

$${F}_{s}^{τ} = 1 - ( 1 - {P}_{{θ}_{T}^{s}}^{L , T} ( {d}_{L} ( {x}_{L}^{T} , {x}_{L}^{T †} ) = 0 ) ) {𝔼}_{{x}_{L}^{T} ∼ {P}_{{θ}_{T}}^{L , T}} [ {d}_{L} ( {x}_{L}^{T} , {x}_{L}^{T †} ) \vert {d}_{L} ( {x}_{L}^{T} , {x}_{L}^{T †} ) > 0 ]$$

Since simulations generated by the forward sampler are highly likely to have non-zero Wasserstein distance to the ground-truth, rejection sampling may be used to estimate the conditional expectation in the last right-hand side term of this equation. This can be done through generating R samples x_{L}^{T,s,r}, with the added non-zero Wasserstein distance constraint. To estimate the scaling factor,

$${P}_{{θ}_{T}^{s}}^{L , T} ( {d}_{L} ( {x}_{L}^{T} , {x}_{L}^{T †} ) = 0 ) ,$$

a coalescent sampler may be used since, by definition, all samples from [symbol] (.|x_{L}^{T†}, θ) have zero Wasserstein distance to the ground-truth. R further coalescent samples \tilde{x}_{L}^{s,r} may thus be generated.

Assuming the probability of each sample under the forward [symbol] and coalescent [symbol] distributions can be calculated, the scaling factor can be estimated using importance sampling:

$$\begin{matrix}{P}_{{θ}_{τ}^{s}}^{L , T} ( {d}_{L} ( {x}_{L}^{T} , {x}_{L}^{T †} ) = 0 ) = \frac{}{} \\ = [ \frac{{{θ}_{τ}^{s}}_{} ( {x}_{L} )}{{{θ}_{τ}^{s}}_{} ( {x}_{L} ) )} ] \\ \approx \frac{1}{R} {\sum }_{r} exp ( log {{θ}_{τ}^{s}}_{} ( {\tilde{x}}_{L}^{s , r} ) - log {{θ}_{τ}^{s}}_{} ( {\tilde{x}}_{L}^{s , r} ) )\end{matrix}$$

where [symbol] is the normalization factor for the forward distribution conditioned on observing zero Wasserstein distance with the ground-truth, and [symbol] is the normalization factor for the coalescent distribution (which we assume to be 1, since the probability of each sample under [symbol] can be calculated).

A multilevel coalescent process [symbol] (.|x_{L}^{T†}, θ) can be defined as follows. For each time-step in descending order, t=(T−1) . . . 0, a focal level l∈{0 . . . L−1} is selected. The N^{L-l} meta-populations at level l, at time t+1, are cycled. For each, a parent meta-population is assigned at time t, independently. A state is assigned to each of the parent, populations, xₗ^{t,j}. If xₗ^{t,j} has one or more children, one of the children xₗ^{t+1,k} is chosen at random. Then, the state of the parent is chosen by sampling xₗ^{t,j}˜Δₗ(.|xₗ^{t+1,k}). However, if xₗ^{t,j} has no children, its state is left undefined.

Not all of the children may have a defined state; if only some children have defined states, xₗ^{t+1,k} is restricted to these cases, and in cases where the child's state is itself not fully defined (for instance, if only some of the members of a meta-population have been defined), the operator Δₗ(.|xₗ^{t+1,k}) is extended to condition on partial states, for example using as many individuals as are defined at each level to calculate the multi-level Wasserstein distance, while preserving the number of defined individuals at each level. between xₗ^{t+1,k} and xₗ^{t,j}. When the coalescent sampler has reached time t=0, any undefined l=0 individuals at t=0 have their states sampled from the initial distribution, and remaining undefined states at t>0 are sampled by forward sampling the multilevel Wright-Fisher process with parameters θ, while maintaining the level assignments at each time-step used in the backward sampling phase, and conditioning on all those individuals whose states have already been assigned in the backwards pass.

Simultaneous Perturbation Stochastic Perturbation (SPSA) provides a method for performing stochastic gradient descent on a function which is easy to evaluate, but for which an analytic gradient is not available. The method requires hyperparameters a, A, α, c, γ, and for epoch τ we define a_{τ}=(a/(τ+A))^{α} and c_{τ}=(c/τ)^{γ}. Then, θ is initialized randomly at epoch 0, and for each epoch τ we sample a vector δ∈{1,1}^{D}^{_{θ}}, representing randomized perturbations to the current value of θ (where δ(i) is sampled independently and uniformly from {−1,1}). To minimize the function F(θ), the following gradient estimator is used at each epoch:

$${\hat{g}}_{τ} = \frac{F ( {θ}_{τ} + {c}_{τ} δ ) - F ( {θ}_{τ} - {c}_{τ} δ )}{2 {c}_{τ}}$$

$${θ}_{τ + 1} = {θ}_{τ} - {a}_{τ} {\hat{g}}_{τ} .$$

The expected Wasserstein distance is used as a function of interest to train the

model, which may be approximated using R Monte-Carlo samples:

$$F ( θ ) = {𝔼}_{{x}_{L}^{T} ∼ {P}_{θ}^{L , T}} [ {d}_{L} ( {x}_{L}^{T} , {x}_{L}^{T †} ) ] \approx \frac{1}{R} {\sum }_{r} {d}_{L} ( {x}_{L}^{T , r} , {x}_{L}^{T †} )$$

Further, coalescent simulations may be incorporated to provide an alternative

estimator of F(θ) based on the following equivalent expression for F(θ):

$$F ( θ ) = ( 1 - {P}_{θ}^{L , T} ( {d}_{L} ( {x}_{L}^{T} , {x}_{L}^{T †} ) = 0 ) ) {𝔼}_{{x}_{L}^{T} ∼ {P}_{θ}^{L , T}} [ {d}_{L} ( {x}_{L}^{T} , {x}_{L}^{T †} ) \vert {d}_{L} ( {x}_{L}^{T} , {x}_{L}^{T †} ) > 0 ] .$$

To estimate θ, given a ground-truth sample x_{L}^{T†} representing a final meta-population at time-step T, the meta-population states at time-steps t<T may be treated as latent variables. An expectation maximization-style approach may be used to train θ by alternately estimating the posterior over x_{L}^{0 . . . T-1} and updating the estimate of θ conditional on this posterior. In a Monte-Carlo Expectation Maximization approach (MC-EM), the posterior over x_{L}^{0 . . . T-1} is represented by sampling. Since it is not possible to directly sample from the required posterior, importance sampling is used to represent it, by performing multiple forward and/or coalescent simulations using the current estimate for θ.

Hence, at epoch τ, for the forward sampling case, S forward simulations are run by sampling from

$${P}_{{θ}_{τ}}^{L , 0 … T} ,$$

where θ_{τ} is the estimate for θ at epoch τ. The term x_{L}^{s} denotes the s^{th} simulation, where the final time-step of each simulation has been replaced by the ground-truth state x_{L}^{T†} (while maintaining all ancestral relationships in the generated sample). During the M-step, θ is updated by optimizing the following importance sampling estimate for the evidence lower bound (ELBO) objective:

$${L}_{1} ( {θ}_{τ + 1} ) = {\sum }_{s} \frac{{P}_{{θ}_{τ}}^{L} ( {x}_{L}^{s} )}{{P}_{{θ}_{τ}}^{L , 0 … T - 1} ( {x}_{L}^{s} ) \cdot {K}_{1}} log {P}_{τ + 1}^{L} ( {x}_{L}^{s} )$$

$${K}_{1} = {\sum }_{s} \frac{{P}_{{θ}_{τ}}^{L} ( {x}_{L}^{s} )}{{P}_{{θ}_{τ}}^{L , … T - 1} ( {x}_{L}^{s} )}$$

To incorporate coalescent simulations into the MC-EM optimizer, s=1 . . . S is drawn forward and r=1 . . . R coalescent simulations (\tilde{x}_{L}^{r}) are drawn at each epoch. The following Monte-Carlo estimator is used for the ELBO bound:

$${L}_{2} ( {θ}_{τ + 1} ) = \frac{{L}_{1}}{2} + {\sum }_{r} \frac{{P}_{{θ}_{τ}}^{L} ( {\tilde{x}}_{L}^{r} )}{2 \cdot {{θ}_{τ}}_{} ( {\tilde{x}}_{L}^{r} ) \cdot {K}_{2}} log {P}_{τ + 1}^{L} ( {\tilde{x}}_{L}^{r} )$$

$${K}_{2} = {\sum }_{r} \frac{{P}_{{θ}_{τ}}^{L} ( {\tilde{x}}_{L}^{r} )}{{{θ}_{τ}}_{} ( {\tilde{x}}_{L}^{r} )}$$

A genetic algorithm can be learned using a multilevel Wasserstein objective. In some embodiments, this approach is used to learn an algorithm to propose solutions to a discrete optimization problem, such as the Traveling Salesman Problem, which may be used for applications in logistics, such as scheduling and planning. Parameters of a fitness function may be learned for a discrete Genetic Algorithm (GA), whose genotypes represent solutions to a Traveling Salesman Problem (TSP), with a prior over the parameter space, where a multilevel Wasserstein distance is used as the objective. This corresponds to a situation in which some ‘good’ solutions to the problem are known, and the underlying cost function and/or an effective mutation policy must be learned to generate further solutions which may improve on those already known.

An underlying TSP consists of finding the shortest path connecting M cities, whose coordinates sampled from a standard two-dimensional Gaussian distribution. Since the coordinates of the cities implicitly define the fitness of a chosen path, the m^{th} city's coordinates are θₘ=(θ_{m,1}, θ_{m,2}), and the goal in training the GA is to learn θ, hence the coordinates of the cities. Further, the distance between cities m₁ and m₂ is represented as d_{m1,m2}. The underlying space of genotypes [symbol] is thus the set of permutations on M elements, and the fitness of genotype x∈ [symbol] is defined as:

$$f ( x ) = exp ( - β {\sum }_{i = 1 … M - 1} {d}_{{x}_{i} , {x}_{i + 1}} )$$

where β is an inverse temperature parameter. A simulation of the GA includes sampling a population of genotypes of size N uniformly from [symbol] , and updating these for T generations using an update rule derived from the Wright-Fisher process with constant population size and selection. Hence, at time-step t>0, to generate a new population of size N, N parents are sampled from the population at t−1, with each parent being sampled with a probability proportional to its fitness (with replacement). The genotypes at generation t are then generated either by coping the parent's genotype, or with probability pₘ, mutating the parents genotype by exchanging two cities (note that reproduction here is asexual, hence each offspring has a single parent).

For the case that the method is to be tested on synthetic data, L simulations are run as above up to time-step T, and a training set is generated by including only the final population state from each simulation. The intention here is to mimic the situation where only genotypes from existing species are available in evolutionary data, or genomics data from a cancer are only available from the time of resection, and the evolutionary history must be inferred. A ground-truth training set may thus be represented as X∈{1 . . . M}^{LNM}, where Xₗₙₘ represents the city (1 . . . M) visited by the n'th population member in simulation l at the m'th location on the route. Similarly, for a given parameter setting θ, a new set of simulations, [symbol] (θ)=\tilde{X}_{θ}∈{1 . . . M}^{LNM}, may be generated of the same dimensions. For convenience, the simulator is assumed to be deterministic, which may be achieved by fixing the random seed. Further, the parameters β, T, pₘ are treated as given, although in general these may also be inferred. Inference is performed using an objective of the form:

F=F₁( [symbol] (θ), [symbol] )
where F₁ may be defined using the multilevel Wasserstein distance as follows:

$${F}_{1} ( \tilde{X} , X ) = exp ( - {W}^{′} ( \tilde{X} , X ) )$$

$${W}^{′} ( \tilde{X} , X ) = {W}_{2} ( \frac{{\sum }_{l} δ ( {\tilde{X}}_{l} )}{L} , \frac{{\sum }_{l} δ ( {\tilde{X}}_{l} )}{L} , C )$$

$$C ( {ρ}_{a} , {ρ}_{b} ) = {W}_{2} ( {ρ}^{a} , {ρ}^{b} , H )$$

where W₂(Pₐ, P_{b}, C) is the second-order Wasserstein distance between distributions Pa and P_{b} using cost function C, ρₐ and ρ_{b} are distributions over genotype space [symbol] , δ(a) is a delta distribution centered at a, and H is the Hamming distance between genotypes, H(xₐ, x_{b})=Σₘ[xₐₘ≠x_{bm}]. Variational optimization or stochastic gradient-based SPSA may be applied to learn the parameter vector θ, where F(θ) is defined identically for each. Further, testing may include 100 meta-epochs for both variational optimization and SPSA algorithms, which is generally sufficient for both algorithms to converge.

In another embodiment of the method, the approach may be used to learn a model of tumor clonal evolution, which may then be applied to predict the subclonal structure of patient tumors from a population and tumor type of interest (for instance, non-small cell lung cancer, in a population of interest). Each run of a genetic algorithm will produce a series of population states ρ₁ . . . ρ_{T}, where each state is a distribution over an underlying space [symbol] representing the genomic state associated with individual tumor cells. The genomic state may be defined as the genotype of the tumor cell (represented as a collection of somatic variants), and/or the transcriptomic/epigenetic state of the tumor cell. The genetic algorithm itself may then be associated with the distribution, at a given time-point T, over population states:

$${P}_{GA} ( {ρ}^{′} ) = \frac{1}{R} {\sum }_{r} [ {ρ}_{T} ( r ) = {ρ}^{′} ]$$

where r summarizes all the sources of randomness in the genetic algorithm, assumed to be generated from a discrete space of size R.

A set of N training samples ρ_{T}¹, . . . , ρ_{T}^{N} from ground truth tumor samples, P_{GA}^{gt}(ρ), may be used to represent the final states of N tumors. The task is to learn a model p_{GA}^{approx}(ρ) from this data. For a given candidate model, the recursive Wasserstein distance may be used as an objective, which in this embodiment may be determined by drawing M samples ρ′_{T}¹, . . . , ρ′_{T}^{M}˜P_{GA}^{approx}(.) using the following formulation:

$${W}_{2} ( {P}_{GA}^{gt} , {P}_{GA}^{approx} , C ) \approx {W}_{2} ( \sum δ ( {ρ}_{T}^{n} ) , {\sum }_{m} δ ( {{ρ}^{′}}_{T}^{m} ) , C )$$

$$C ( {ρ}^{a} , {ρ}^{b} ) = {W}_{2} ( {ρ}^{a} , {ρ}^{b} , {C}^{x} )$$

Where W₂ is the second-order Wasserstein distance, as above, and [symbol] is a distance matrix on the space [symbol] of tumor cell genomic states (incorporating genetic and/or transcriptomic/epigenetic information). This is a two-level objective function implementation of the more general multilevel problem described above.

For the case that genetic and transcriptomics data are used to train the model, the model may be expressed as:

$$P ( {z}_{i} , 0 ) ∼ \frac{1}{{2}^{{N}_{snp}}}$$

$${x}_{i , t} ∼ ( \cdot \vert {z}_{i , t}^{T} \cdot {θ}_{g} , {σ}_{g}^{2} I )$$

$${w}_{i , t} ∼ {e}^{{x}_{i , t}^{T} \cdot {θ}_{f}}$$

$${ω}_{it} = \frac{{w}_{it}}{{\sum }_{j} {w}_{jt}}$$

$${π}_{i , t} ∼ Categorical ( \cdot \vert [ {ω}_{1 , t - 1} , … , {ω}_{N , t - 1} )$$

$${z}_{i , t > 0} ∼ {\prod }_{j = 1 … {N}^{snp}} ( {θ}_{h}^{{z}_{{π}_{i , t} , t - 1 , j \neq {z}_{i , t , j}}} \cdot {( 1 - {θ}_{h} )}^{{z}_{{π}_{i , t} , t - 1 , j = {z}_{i , t , j}}} )$$

where z, x, w, π represent genotypes (SNPs) transcriptome (gene expression), fitness, and evolutionary-tree respectively, while θ_{f}, θ_{g}, and θₕ are gene fitness, SNP effect, and mutation rate parameters respectively, with θₕ being fixed above. Here, z_{i,t}, is the genotype of the i'th cell in the population at time t, x_{i,t} is the transcriptome of the i'th cell in the population at time t, θ_{g} is a matrix of SNP effects on gene expression, θ_{f} is a matrix of gene effects on fitness, w_{i,t} and Ωᵢₜ are respectively the absolute and relative fitness of the i'th cell in the population at time t, π_{i,t} denotes the parent of the i'th cell in the population at time t in the population at time t−1, and Categorical(.|μ) is a categorical distribution with mean μ. The equations above define a Wright-Fisher model with fitness over genotypes and transcriptomes, which may be sampled to produce forward samples from the model given θ_{f}, θ_{g}, and θₕ. Further, while the parameters θ_{f}, θ_{g}, and θₕ determine linear predictors for fitness, gene expression and the mutation rate in the model above, these may also represent the parameters of single or multi-layer neural networks, hence determining non-linear predictors of these respective variables.

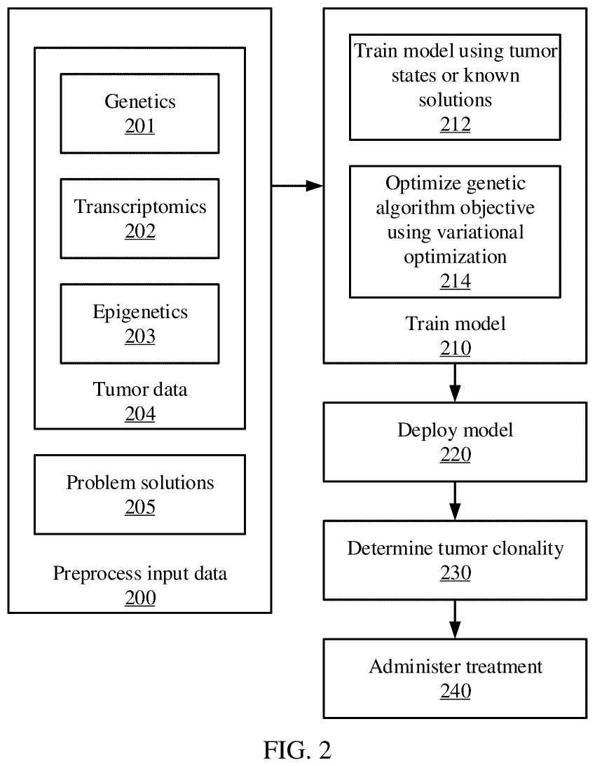

Referring now to FIG. 2, a method of training and using a machine learning model is shown. Block 200 preprocesses the input data. For the tumor clonality embodiment, this data involves genomics tumor data of the kinds shown in block 204, which may include genetics 201 (for instance, somatic variant data formatted as VCF files), transcriptomics 202 (for instance, single-cell read count data) and/or epigenetics 203 (for instance, single-cell ATACseq data). This data should be single-cell level data, generated from a cohort of tumors (e.g. lung cancer patients undergoing a common treatment), each generating a population of cell-level descriptor vectors (the ‘genotypes’ on which the genetic algorithm acts). For the discrete optimization embodiment, this data involves a (meta-) population of known solutions or approximate solutions to a target optimization problem (for instance, approximate TSP solutions), as in block 205. Block 210 trains the model, for example using a genetic algorithm as described above. The training data may include training samples from the final states of N tumors, or known (approximate) solutions to an optimization problem 212. Block 214 optimizes the genetic algorithm objective, such as one of the multilevel Wasserstein distance objectives above, using variational optimization or any other appropriate optimization method. In the process, the training may optimize the parameters of neural networks associated with fitness, mutation and gene expression predictors.

Once the model has been trained, block 220 deploys the model to a target environment. In some cases, where the training is performed in the same system that performs subsequent inference, the deploying may be omitted. In other cases, deploying may include transmitting the parameters of the trained model to the target environment. For the tumor clonality embodiment, Block 230 then uses the deployed model for inference, for example to determine the clonality of some new tumor. This inference may be performed by drawing samples from the multilevel coalescent process defined above, with the trained parameters (for instance, neural network parameters of the fitness, mutation and gene expression models) applied to a new target population; alternatively, samples may be drawn from the trained forward model, and an optimal one-to-one mapping found between the sample with the smallest Wasserstein distance to the target tumor, to estimate the subclonal structure of the new tumor. The clonality information generated by the model, which may be expressed as a tree sampled from the forward or coalescent model as described above, along with subclonal properties such as subclone fitness, subclonal shared variants and subclonal gene expression, is used in block 240 to administer a treatment to a patient. The administration of a treatment may include automatically administering a therapy to a patient (e.g., a tailored pharmaceutical) and may also include generating a report for medical professionals to aid in decision making.

A given tumor may have a number of genetic variants in it that have non-neutral effects. The trained model may be used to infer the evolutionary history of a specific tumor, such as one that is identified within a patient. The model learns generative models of natural evolutionary processes, such as those occurring in cancer, to identify key features such as driver variants and tumor clonal structure. These methods can be extended to evolutionary models with arbitrary numbers of layers, identifying the number of evolutionary levels in a system and analyzing the interaction and emergence of units at multiple levels. In providing a treatment, the genetic variants within a tumor which drive its replication may be targeted to tailor a treatment that is specific to the cancer that the individual patient has; this may be achieved by targeting the subclones with the highest fitness values according to the trained fitness model.

Alternatively, in a discrete optimization embodiment, the deployed model may be used to generate novel solutions to the discrete optimization problem (for example, TSP solutions), which may then be used for scheduling and/or planning purposes.

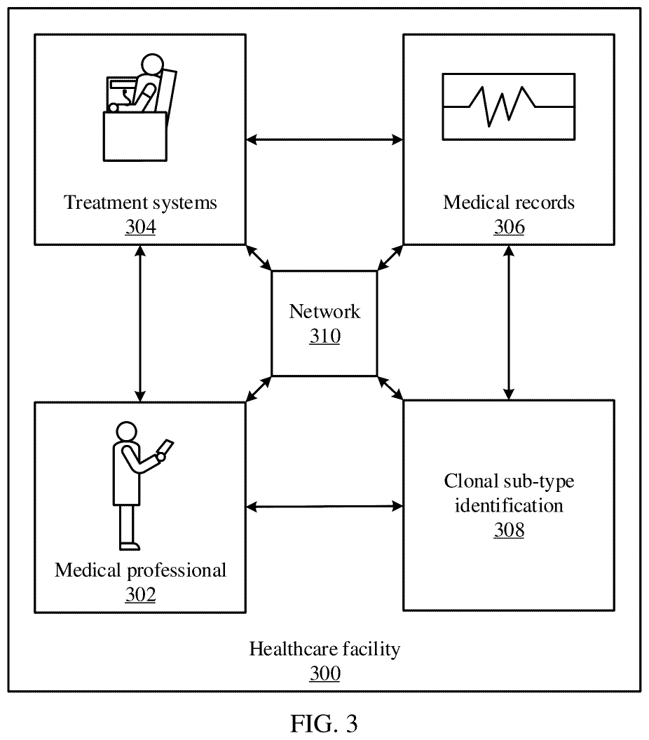

Referring now to FIG. 3, a diagram of information extraction is shown in the context of a healthcare facility 300. Clonal sub-type identification 308 may be used to determine the genetic sub-types that make up a tumor that has been found within a patient, for example based on a tissue sample analysis in medical records 306.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed. For example, the treatment systems 304 may include monitoring sensors that take measurements of the patient's medical status and may further include automated treatment systems that may automatically apply a treatment. An example of such an automated treatment system includes an automatically triggered intravenous drug delivery system.

Based on information provided by the clonal sub-type identification 308, the medical professionals 302 may make medical decisions about patient healthcare suited to the patient's needs. For example, the medical professionals 302 may make a diagnosis of the patient's health condition and may prescribe particular medications, surgeries, and/or therapies. In some cases the therapies may be tailored to the specific clonal sub-types identified in the tumor.

The different elements of the healthcare facility 300 may communicate with one another via a network 310, for example using any appropriate wired or wireless communications protocol and medium. Thus the clonal sub-type identification 308 can be used to design a treatment that targets a patient's specific condition, for example using tissue samples and medical records 306. The treatment systems 304 may be used to generate and administer a therapy based on the identified clonal sub-types of a patient's tumor.

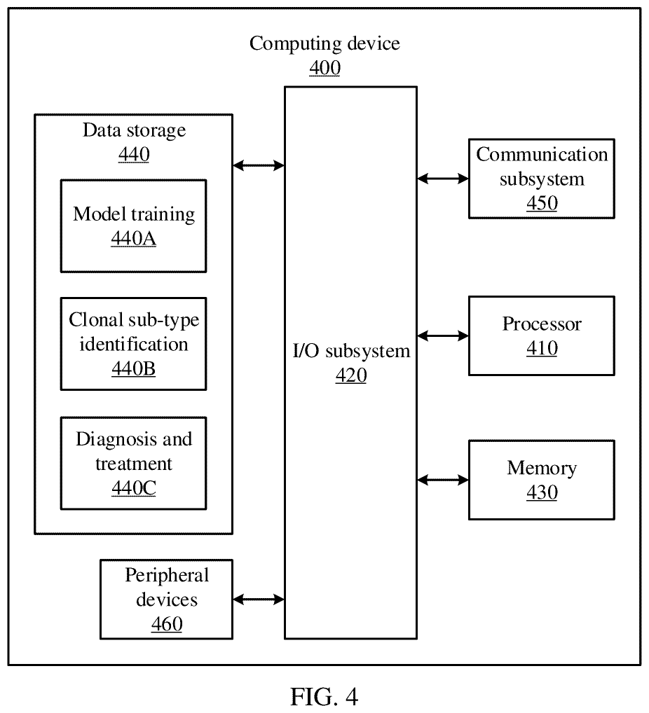

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for training a model, 440B for clonal sub-type identification, and/or 440C for generating a treatment based on a patient's condition. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

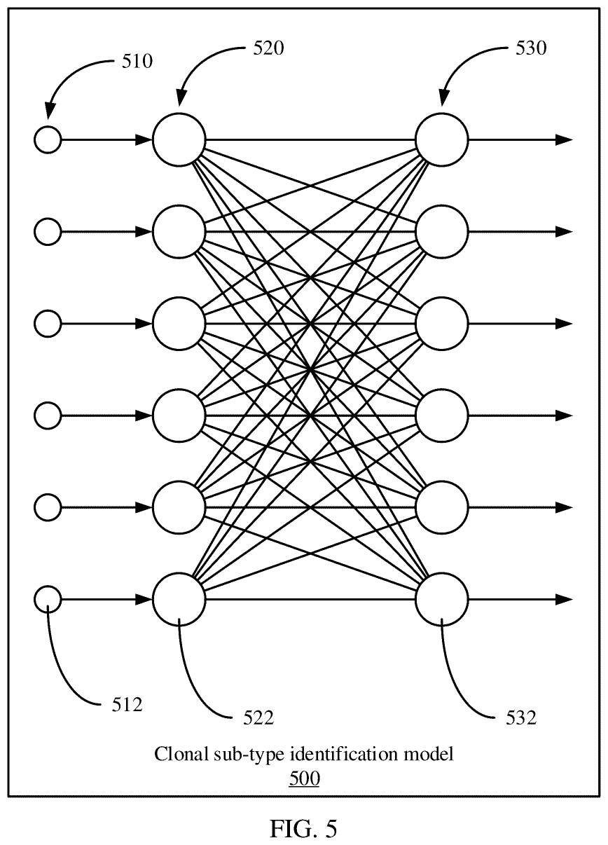

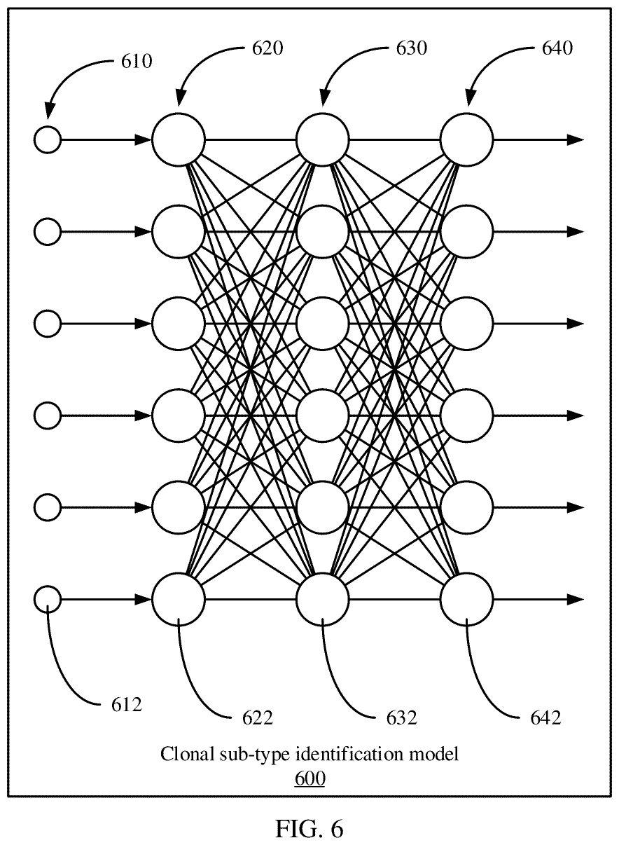

Referring now to FIGS. 5 and 6, exemplary neural network architectures are shown, which may be used to implement parts of the present models, such as the fitness, gene expression and mutation rate predictors described above 500/600. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
analyzing genotypes of a tumor to identify clonality sub-types present in the tumor, using a machine learning model that is trained to learn a multilevel evolutionary process or genetic algorithm, by using a recursive Wasserstein objective to output the clonal sub-types, an ancestral structure, and a fitness model; and
generating a treatment tailored to the tumor using the clonal sub-types.

2. The method of claim 1, further comprising training the machine learning model using the recursive Wasserstein objective based on training data that includes final tumor states, including single-cell level genetics, transcriptomics and epigenetic measurements.

3. The method of claim 2, wherein the recursive Wasserstein objective is evaluated as
$${W}_{2} ( \sum δ ( {ρ}_{T}^{n} ) , {\sum }_{m} δ ( {{ρ}^{′}}_{T}^{m} ) , C )$$
where W₂(⋅) is a second-order Wasserstein distance, δ(⋅) is a delta distribution, ρ_{T}ⁿ is a sample from the training data, p′_{T}^{m} is a sample from the model, and
C(ρ^{a}, ρ^{b})=W₂(ρ^{a}, ρ^{b}, [symbol] )
where [symbol] is a distance matrix on the space of genotypes, [symbol] .

4. The method of claim 1, wherein training the machine learning model includes optimizing the recursive Wasserstein objective using an optimization technique selected from the group consisting of variational optimization, simultaneous perturbation stochastic perturbation, and Monte-Carlo expectation maximization.

5. The method of claim 1, further comprising generating a report for medical professionals to be used in treatment decision making.

6. The method of claim 1, further comprising automatically administering the treatment to a patient with the tumor.

7. The method of claim 6, wherein automatically administering the treatment to the patient includes triggering an automated intravenous drug delivery system.

8. The method of claim 6, further comprising monitoring a medical status of the patient to inform the treatment.

9. The method of claim 1, wherein the machine learning model used for the fitness, gene expression, and mutation rate predictors is a neural network model.

10. The method of claim 1, wherein the tumor includes a plurality of clonal sub-types that represent genetic variants of cells within the tumor.

11. A system, comprising:
a hardware processor; and
a memory that stores a computer program which, when executed by the hardware processor, causes the hardware processor to:
analyze genotypes of a tumor to identify clonality sub-types present in the tumor, using a machine learning model that is trained to learn a multilevel evolutionary process or genetic algorithm, by using a recursive Wasserstein objective to output the clonal sub-types, an ancestral structure, and a fitness model; and
generate a treatment tailored to the tumor using the clonal sub-types.

12. The system of claim 11, wherein the computer program further causes the hardware processor to train the machine learning model using the recursive Wasserstein objective based on training data that includes final tumor states, including single-cell level genetics, transcriptomics and epigenetic measurements.

13. The system of claim 12, wherein the recursive Wasserstein objective is evaluated as
$${W}_{2} ( \sum δ ( {ρ}_{T}^{n} ) , {\sum }_{m} δ ( {{ρ}^{′}}_{T}^{m} ) , C )$$
where W₂(⋅) is a second-order Wasserstein distance, δ(⋅) is a delta distribution, ρ_{T}ⁿ is a sample from the training data, p′_{T}^{m} is a sample from the model, and
C(ρ^{a}, ρ^{b})=W₂(ρ^{a}, ρ^{b}, [symbol] )
where [symbol] is a distance matrix on the space of genotypes, [symbol] .

14. The system of claim 11, wherein the computer program further causes the hardware processor to optimize the recursive Wasserstein objective using an optimization technique selected from the group consisting of variational optimization, simultaneous perturbation stochastic perturbation, and Monte-Carlo expectation maximization.

15. The system of claim 11, wherein the computer program further causes the hardware processor to generate a report for medical professionals to be used in treatment decision making.

16. The system of claim 11, wherein the computer program further causes the hardware processor to automatically administer the treatment to a patient with the tumor.

17. The system of claim 16, wherein automatic administration of the treatment to the patient includes triggering an automated intravenous drug delivery system.

18. The system of claim 16, wherein the computer program further causes the hardware processor to monitor a medical status of the patient to inform the treatment.

19. The system of claim 11, wherein the machine learning model used for the fitness, gene expression, and mutation rate predictors is a neural network model.

20. The system of claim 11, wherein the tumor includes a plurality of clonal sub-types that represent genetic variants of cells within the tumor.
